(19) United States
(12) Patent Application Publication
(10) Pub. No.: US 2004/0147777 A1
(43) Pub. Date: Jul. 29, 2004

(54) Method for oxidising hydrocarbons

(76) Inventors: Eric Fache, Caluire et Cuire (FR); Jean-Pierre Simonato, Sassenage (FR)

Correspondence Address: BURNS DOANE SWECKER & MATHIS L L P, POST OFFICE BOX 1404, ALEXANDRIA, VA, 22313-1404, US

(21) Appl. No.: 10/476,533

(22) PCT Filed: Apr. 30, 2002
(86) PCT No.: PCT/FR02/01492

(30) Foreign Application Priority Data
May 4, 2001 (FR) 01/06016

(51) Int. Cl. C07C 51/255

(52) U.S. Cl. 562/412; 562/452

(57) ABSTRACT

The present invention relates to a process for the oxidation of hydrocarbons, in particular of branched or unbranched saturated aliphatic hydrocarbons or of cycloaliphatic or alkylaromatic hydrocarbons, to alcohol, ketone and/or acid or polyacid compounds.

It relates more particularly to the oxidation, by an oxidizing agent comprising molecular oxygen, of cyclohexane to cyclohexanonol, cyclohexanone and/or adipic acid. The oxidation is carried out in the presence of a catalytic system comprising a catalyst based on at least one metal compound and a cocatalyst comprising an imide functional group, such as N-bromosuccinimide, N-bromomaleimide, N-bromohexahydrophthalimide, N,N′-dibromocyclohexane-tetracarboximide, N-bromophthalimide, N-bromotrimellitimide or N,N′-dibromopyromellitimide.

The present invention relates to a process for the oxidation of hydrocarbons, in particular of branched or unbranched saturated aliphatic hydrocarbons or of cycloaliphatic or alkylaromatic hydrocarbons, to alcohol, ketone and/or acid or polyacid compounds.

It relates more particularly to the oxidation, by an oxidizing agent comprising molecular oxygen, of cyclohexane to cyclohexanonol, cyclohexanone and/or adipic acid.

The oxidation of cyclohexane to adipic acid is a process which has been studied for many years. This is because adipic acid is an important chemical compound used as starting material in numerous manufacturing operations, such as the production of polymers, for example polyamides, polyesters or polyurethanes.

Several processes for the manufacture of adipic acid from hydrocarbons, such as benzene, phenol, cyclohexene or cyclohexane, have been proposed.

The oxidation of the cyclohexane, either directly or in two stages, are the most advantageous routes for producing adipic acid.

Thus, U.S. Pat. No. 2,223,493, published in December 1940, discloses the oxidation of cyclic hydrocarbons to corresponding diacids, in a liquid phase generally comprising acetic acid, at a temperature of at least 60° C., using a gas comprising oxygen and in the presence of an oxidation catalyst, such as a cobalt compound.

Numerous other patents and articles disclose this direct oxidation reaction of cyclohexane to adipic acid. However, to obtain acceptable yields for the production of adipic acid, these documents disclose the use of acetic acid as solvent, in the presence either of a homogeneous catalyst or of a heterogeneous catalyst. Mention may be made, by way of illustration, of the article which appeared in the journal “Chemtech”, 555-559 (September 1974), the author of which is K. Tanaka, which summarizes and comments upon the process for the direct oxidation of cyclohexane. Mention may also be made of U.S. Pat. Nos. 3,231,608, 4,032,569, 4,158,73, 4,263,453 and 5,321,157 and European Patent 870751, which disclose various homogeneous catalytic systems.

A few processes for the oxidation, in a single stage, of cyclohexane to adipic acid without the use of acetic acid have also been proposed. Some propose to carry out this reaction in the absence of solvents, others with solvents such as organic esters, for example acetates (U.S. Pat. No. 4,098,817), acetone (U.S. Pat No. 2,589,648), alcohols, such as butanol, methanol or cyclohexanol, or acetonitrile (EP 784 045).

The oxidation of cyclohexane to cyclohexanone and/or cyclohexanol is also an important industrial process as these compounds are important chemical intermediates in the synthesis of numerous products. This oxidation also constitutes the first stage of the process for the manufacture of adipic acid, the second stage being an oxidation of the cyclohexanone/cyclo-hexanol mixture by nitric acid. Furthermore, cyclohexanone is an important starting material in the manufacture of ε-caprolactam, the monomer of polymamide 6.

Numerous patents and publications have disclosed the oxidation of cyclohexane to cyclohexanone/cyclohexanonol by oxidation by oxygen or a gas comprising oxygen. Cyclohexanol is converted to cyclohexanone by a dehydrogenation reaction, as described in “Handbook of Chemistry; Applied Chemistry”, page 356, 1986 version, published by Nippon Kagaku-Kai.

The catalytic systems used for these oxidation reactions are generally based on a metal compound, such as chromium, cobalt, iron, nickel, cerium, zirconium or manganese compounds.

To improve the activity of these catalysts, the proposal has in particular been made to add a compound comprising imide functional groups, more particularly N-hydroxyphthalimide, as disclosed in European Patents 1 074 537, 824 962 and 1 074 536, for example.

One of the aims of the present invention is to provide a process for the oxidation of hydrocarbons by oxygen or a gas comprising oxygen in the presence of a catalytic system comprising a catalyst based on a metal compound and of a cocatalyst which makes it possible to improve the activity of the catalyst based on a metal compound without decreasing the selectivity for the desired oxidation product or products, more particularly for ketone, alcohol and/or carboxylic acids.

To this end, the invention provides a process for the oxidation of substituted or unsubstituted saturated aliphatic or cycloaliphatic hydrocarbons or of alkylaromatic hydrocarbons by an oxidizing agent comprising molecular oxygen, characterized in that the oxidation is carried out in the presence of a catalytic system comprising a catalyst based on at least one metal compound and a cocatalyst comprising an imide functional group and corresponding to one of the following general formulae:

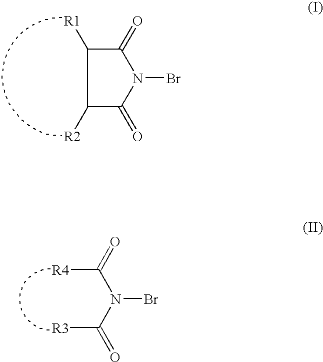

in which:

R1 and R2, which are identical or different, can be hydrogen, an aliphatic, aromatic, cycloaliphatic, arylaliphatic or alkylaromatic hydrocarbonaceous radical which comprises from 1 to 12 carbon atoms and which can comprise heteroatoms, a halogen atom, a hydroxyl group, an alkoxy group, a carboxyl group, an ester group or a carbonyl group, it being possible for the radicals R1 and R2 to be connected to one another to form a cycloaromatic radical, which can comprise several aromatic rings in the condensed or uncondensed form, or a cycloaliphatic radical, which can comprise one or more rings in the condensed or uncondensed form,

R3 and R4, which are identical or different, can be hydrogen or an aliphatic, aromatic, cycloaliphatic, arylaliphatic or alkylaromatic hydrocarbonaceous radical which comprises from 1 to 20 carbon atoms and which can comprise heteroatoms, it being possible for the radicals R3 and R4 to be connected to one another to form a cycloaromatic radical, which can comprise several aromatic rings in the condensed or uncondensed form, or a cycloaliphatic radical, which can comprise one or more rings in the condensed or uncondensed form.

The oxidation reaction can be carried out in the gas phase or in the liquid phase.

In one embodiment, the oxidation reaction is carried out in order to preferably obtain, as oxidation products, alcohols and/or ketones. In this embodiment, the solvent used is advantageously the hydrocarbon to be oxidized. However, it is possible to use other solvents, such as other non-oxidizable hydrocarbons, nitrites, esters, aromatic derivatives or alcohols.

In another embodiment of the invention, the oxidation of the hydrocarbon is carried out in order to directly obtain an acid or polyacid. In this embodiment, it is preferable to use a solvent chosen from carboxylic acids, such as acetic acid, glutaric acid, octanoic acid or lipophilic acids in general. Such solvents are already described.

Suitable lipophilic acid compounds is understood to mean aromatic, aliphatic, arylaliphatic or alkylaromatic organic compounds which comprise at least 6 carbon atoms, which can comprise several acidic functional groups and which exhibit a low solubility in water, that is to say a solubility of less than 10% by weight at ambient temperature (1020 C.; 30° C.).

Mention may be made, as lipophilic organic compound, of, for example, hexanoic acid, heptanoic acid, octanoic acid, 2-ethylhexanoic acid, nonanoic acid, decanoic acid, undecanoic acid, dodecanoic acid or stearic (octadecanoic) acid and their permethylated derivatives (complete substitution of the hydrogens of the methylene groups by the methyl group), 2-octadecyl-succinic acid, 2,5-di (tert-butyl)benzoic acid, 4-(tert-butyl)benzoic acid, 4-octylbenzoic acid, tert-butyl hydrogen orthophthalate, naphthenic or anthracenic acids substituted by alkyl groups, preferably of tert-butyl type, substituted phthalic acid derivatives, or fatty diacids, such as fatty acid dimer. Mention may also be made of acids belonging to the preceding families and carrying various electron-donating substituents (groups with a heteroatom of the O or N type) or electron-withdrawing substituents (halogens, sulphonimides, nitro or sulphonato groups or the like).

According to another characteristic of the invention, the concentration of acid compound in the reaction medium is specified so as to obtain a molar ratio of the number of moles of acid to the number of moles of metal forming the catalyst of between 0.5 and 1 000 000, preferably between 1 and 100 000.

The concentration of acid compound in the liquid oxidation medium can vary within wide limits. Thus, it can be between 1 and 99% by weight with respect to the total weight of the liquid medium and more advantageously it can be between 10 and 80% by weight of the liquid medium.

It is also possible, without in doing so departing from the scope of the invention, to use the acid component in combination with another compound which can in particular have the effect of improving the productive output and/or the selectivity of the oxidation reaction for adipic acid, and in particular the dissolution of oxygen.

Mention may in particular be made, as examples of such compounds, of nitrites or halogenated compounds, more advantageously fluorinated compounds. Mention may be made, as compounds which are more particularly suitable, of nitrites, such as acetonitrile or benzonitrile, halogenated derivatives, such as dichloromethane, or fluorinated compounds, such as:

cyclic or acyclic, fluorinated or perfluorinated aliphatic hydrocarbons, or fluorinated aromatic hydrocarbons, such as perfluorotoluene, perfluoromethylcyclohexane, perfluorohexane, perfluoroheptane, perfluorooctane, perfluorononane, perfluorodecalin, perfluoromethyldecalin, α,α,α-trifluorotoluene or 1,3-bis (trifluoromethyl) benzene;

perfluorinated or fluorinated esters, such as alkyl octanoates which are perfluorinated or alkyl nonanoates which are perfluorinated;

fluorinated or perfluorinated ketones, such as perfluorinated acetone;

fluorinated or perfluorinated alcohols, such as perfluorinated hexanol, octanol, nonanol or decanol, perfluorinated t-butanol, perfluorinated isopropanol or 1,1,1,3,3,3-hexafluoro-2-propanol;

fluorinated or perfluorinated nitriles, such as perfluorinated acetonitrile;

fluorinated or perfluorinated acids, such as (trifluoromethyl)benzoic acids, pentafluorobenzoic acid, perfluorinated hexanoic, heptanoic, octanoic or nonanoic acid, or perfluorinated adipic acid;

fluorinated or perfluorinated halides, such as perfluorinated iodooctane or perfluorinated bromooctane;

fluorinated or perfluorinated amines, such as perfluorinated tripropylamine, perfluorinated tributylamine or perfluorinated tripentylamine.

In the embodiments of the invention, the catalyst based on a metal compound advantageously comprises a compound of at least one metal element chosen from the group consisting of Cu, Ag, Au, Mg, Ca, Sr, Ba, Zn, Cd, Hg, Al, Sc, In, Tl, Y, Ga, Ti, Zr, Hf, Ge, Sn, Pb, V, Nb, Ta, Cr, Mo, W, Mn, Tc, Re, Fe, Ru, Os, Co, Rh, Ir, Ni, Pd, Pt, lanthanides, such as Ce, and the combinations of these. Compound of a metal element should be understood as meaning the compounds comprising at least one atom of the said metal in combination with other chemical elements, such as, for example, oxygen, but also the metal alone.

These catalytic metal elements are employed either in the form of compounds advantageously at least partially soluble in the liquid oxidation medium under the conditions for implementation of the oxidation reaction, which embodiment is referred to hereinbelow as “homogeneous catalysis”, or are supported on, absorbed on or bonded to, more generally impregnated in, an inert support, such as silica or alumina, for example. This embodiment is referred to hereinbelow as “heterogeneous catalysis”. The latter form of catalyst is suitable in particular for carrying out gas-phase oxidation.

In homogeneous catalysis, the metal catalyst is preferably, in particular under the conditions for carrying out the oxidation reaction:

either soluble in the hydrocarbon to be oxidized,

or soluble in the acid compound used as solvent,

or soluble in the hydrocarbon/acid compound mixture forming a homogeneous liquid phase under the conditions for carrying out the reaction.

According to a preferred embodiment of the invention, the catalyst used is soluble in one of these media at ambient temperature or at the temperature for recycling these media in a further oxidation.

The term “soluble” is understood to mean that the catalyst is at least partially soluble in the medium under consideration.

In the case of heterogeneous catalysis, the catalytically active metal elements are supported on or incorporated in a micro—or mesoporous inorganic matrix or on or in a polymer matrix or are in the form of organometallic complexes grafted to or incorporated in an organic or inorganic support. The term “incorporated” is understood to mean that the metal is an element of the support or that the operation is carried out with complexes or compounds of the catalytically active metal which are sterically and/or chemically trapped within the structure, for example porous structure, of the support under the conditions of the oxidation.

In a preferred embodiment of the invention, the homogeneous or heterogeneous metal catalyst is composed of salts or of complexes of metals from Groups IVb (Ti group), Vb (V group), VIb (Cr group), VIIb (Mn group), VIII (Fe or Co or Ni group) and Ib (Cu group), and cerium, alone or as a mixture. The preferred elements are in particular Co and/or Mn and/or Cr and/or Zr, Hf, Ce and/or Zr and/or Hf. The concentration of metal in the liquid oxidation medium, in homogeneous catalysis, varies between 0.00001 and 5% (% by weight), preferably between 0.00001% and 1%, with respect to the whole of the reaction mass.

According to the invention, the catalytic system comprises a cocatalyst composed of an organic compound defined by the general formulae (I) and (II) described above.

This compound is added to the oxidation medium or can be incorporated in a support in the case where heterogeneous catalysis is carried out, the support advantageously comprising the catalytically active metal. The term “incorporated” has the meaning indicated above.

The molar ratio of the cocatalyst in the oxidation medium can vary within wide limits. By way of example, this ratio can be between 0.001 mol and 1 mol of cocatalyst per one mole of hydrocarbon to be oxidized. Advantageously, this ratio can be between 0.001 mol and 0.2 mol.

Mention may be made, as cocatalysts which are suitable for the invention, of the compounds corresponding to the formula (I) in which R1 and R2, which are identical or different, represent hydrogen or alkyl radicals, such as methyl, ethyl, propyl, isopropyl, butyl, isobutyl, sec-butyl, t-butyl, pentyl, hexyl, heptyl, octyl, decyl, dodecyl or branched radicals. R1 and R2 can also represent an aromatic group, such as the phenyl, benzyl, naphthyl or toluyl groups. Mention may also be made, as radicals represented by R1 and R2, of cycloalkyl radicals, such as cyclohexyl, cyclopentyl or cyclooctyl.

R1 and R2 can also represent, as indicated above, an alkoxycarbonyl or acyl radical. Mention may be made, as preferred radicals, of methoxycarbonyl, ethoxycarbonyl, propoxycarbonyl, isopropoxycarbonyl, butoxycarbonyl, pentoxycarbonyl or the formyl, acetyl, propionyl, butyryl, valeryl or pivaloyl radicals.

In addition, R1 and R2 can be bonded to one another via a single or double bond to form an aromatic or aliphatic ring in the condensed or uncondensed form. These rings can also be aromatic or aliphatic heterocycles. Mention may be made, as examples of rings thus formed, of benzene and naphthene rings for aromatic compounds and cyclohexane and cyclododecane for aliphatic rings.

Mention may be made, as examples of compounds corresponding to the formula (I) which are suitable for the invention, of N-bromosuccinimide, N-bromomaleimide, N-bromohexahydrophthalimide, N,N′-dibromocyclohexane-tetracarboximide, N-bromophthalimide, N-bromotrimellitimide or N,N′-dibromopyromellitimide.

The cocatalysts corresponding to the formula (II) are in particular those in which R3 and R4, which are identical or different, represent the radicals shown for R1 and R2. Mention may be made, as preferred compounds, of the compounds N-bromosuccinimide, N-bromophthalimide or N-bromonaphthalimide.

These compounds are either commercially available, such as, for example, the compounds mentioned above, or can be obtained by a conventional process for formation of an imide, for example by reaction between an acid anhydride and hydroxylamine, followed by reaction with a brominating agent, such as sodium hypobromite or bromite.

The invention applies more particularly to the oxidation of cycloaliphatic compounds, such as cylcohexane or cyclododecane, to corresponding linear diacids or to corresponding alcohols and ketones.

According to a preferred embodiment of the invention, the invention relates to the direct oxidation of cyclohexane to adipic acid by a gas comprising oxygen in a liquid medium and in the presence of a catalyst. The catalyst preferably comprises cobalt or manganese.

The oxidation reaction is carried out at a temperature of between 50° C. and 200° C., preferably between 70° C. and 180° C. It can be carried out at atmospheric pressure. However, it is generally carried out under pressure in order to keep the components of the reaction medium in the liquid form. The pressure can be between 10 kPa (0.1 bar) and 20 000 kPa (200 bar), preferably between 100 kPa (1 bar) and 10 000 kPa (100 bar).

The oxygen used can be in the pure form or in the form of a mixture with an inert gas, such as nitrogen or helium. It is also possible to use air more or less enriched in oxygen. The amount of oxygen fed to the medium is advantageously between 1 and 1 000 mol per mole of compounds to be oxidized.

The oxidation process can be carried out continuously or according to a batchwise process. Advantageously, the liquid reaction medium exiting from the reactor is treated according to known processes which make it possible, on the one hand, to separate and recover the acid produced and, on the other hand, to recycle the unoxidized or partially oxidized organic compounds, such as cyclohexane, cyclohexanol and/or cyclohexanone, the catalytic system and the acid compound used as solvent.

The amount of metal catalyst, expressed as percentage by weight of metal with respect to the reaction mixture, is generally between 0.00001% and 5% and preferably between 0.00001% and 1%, without these values being critical. However, it is a question of having sufficient activity while not using excessively large amounts of catalyst, which subsequently must be separated from the final reaction mixture and recycled.

The metal catalyst, in addition to cobalt and/or manganese, can also comprise other compounds based on metals chosen from the group consisting of manganese, copper, cerium, vanadium, chromium, zirconium, hafnium, cobalt and a combination of some of these elements.

It is advantageous to also employ a compound which initiates the oxidation reaction, such as, for example, a ketone or an aldehyde. Cyclohexanone, which is a reaction intermediate in the case of the oxidation of cyclohexane, is very particularly indicated. Generally, the initiator represents from 0.01% to 20% by weight of the weight of the reaction mixture employed, without these proportions having a critical value. The initiator is useful in particular during the initiation of the oxidation and when the oxidation is carried out at a temperature of less than 120° C. It can be introduced from the beginning of the reaction.

The oxidation can also be carried out in the presence of water introduced from the initial stage of the process.

As indicated above, the reaction mixture resulting from the oxidation is subjected to various operations for the separation of some of its constituents in order, for example, to make it possible to recycle them in the oxidation and to make possible the recovery of the acids produced.

According to a first alternative form of the process, the crude reaction mixture can first of all be subjected to cooling to a temperature of 160° C. to 300° C., for example, which results in the crystallization of at least a portion of the acid formed. A medium comprising a solid phase composed essentially of acids, at least one liquid organic phase essentially comprising the unreacted compound to be oxidized, possibly the acid compound and the oxidation intermediates (or several organic phases if the acid compound and the hydrocarbon are not completely miscible at low temperature), and a liquid aqueous phase essentially comprising acid by-products from the oxidation and the water formed, is thus obtained. The catalytic system can be in one of the organic phases, if it is soluble in the said phase, or in the lower aqueous phase.

After filtering or centrifuging off the solid, the liquid organic and aqueous phases constituting the filtrate or the centrifugate are separated by settling, if necessary: the organic phase or phases can be recycled in a further oxidation reaction.

It can be advantageous, prior to the operation of crystallizing the acid, to concentrate the reaction mixture.

According to a second alternative form of the process, the final crude reaction mixture can be withdrawn under hot conditions, for example at a temperature which can reach 75° C. The reaction mixture then separates by settling into at least two liquid phases: one or more organic phases essentially comprising the unreacted hydrocarbon, possibly the acid compound or the oxidation intermediates, and a liquid aqueous phase essentially comprising the acids formed and the water formed. Depending on the solubility and the nature of the catalytic system, the latter can be present in the organic phase or phases, can be recovered by solid/liquid separation before precipitation or crystallization of the acid formed, in the case of heterogeneous catalysis, or, if it is soluble in the aqueous phase, can be extracted by liquid/liquid extraction through a resin or electrodialysis.

As in the first alternative form, the liquid phases are separated by settling: the organic phase or phases can be recycled in a further oxidation reaction.

In these embodiments, the acid compound used as solvent is generally present in or forms an essential component of the organic phase or phases. Consequently, after separation of the acid formed and optionally of the liquid phase comprising the water formed, the oxidation by-products and the catalyst, the acid compound is recycled in the oxidation stage with the unoxidized hydrocarbon and the oxidation intermediates.

Furthermore, if the acid compound is solid in a phase of treatment of the reaction medium, it will be advantageously separated and recovered by employing solid/liquid separation processes, either before treatment of the reaction medium to recover the acid produced or with the acid produced. In the latter case, the acid produced can be recovered by extraction with water.

In these embodimental examples of the invention, water can be added to the reaction medium in order to obtain better dissolution of the acid by-products from the oxidation and better recovery of the acid formed.

The acid is generally recovered by precipitation during the cooling of the reaction medium. The acid thus recovered can be purified using standard techniques disclosed in numerous patents. Mention may be made, by way of examples, of French Patents Nos. 2 749 299 and 2 749 300.

If the liquid nonorganic or aqueous phase comprises the catalyst, the latter is extracted, either before crystallization of the acid formed, by precipitation or extraction according to known processes, such as liquid-liquid extraction, electrodialysis or treatment through ion-exchange resins, for example, or after crystallization of the acid formed, by extraction techniques described above or similar techniques.

In the embodiment of oxidation of hydrocarbons to alcohols and ketones, the invention advantageously applies to the oxidation of cycloalkanes to cycloalkanones and cycloalkanols, more particularly to the oxidation of cyclohexane to cyclohexanol and cyclohexanone.

In this embodiment, the catalytic systems can be identical to those described for the direct oxidation to acids.

The reaction medium does not comprise a solvent of the acid type, the hydrocarbon to be oxidized, for example cyclohexane, advantageously being the solvent for the reaction products. The operating conditions for carrying out the oxidation reaction are advantageously a temperature of between 130° C. and 200° C. and a pressure of between 1 and 10 bar.

The products from the oxidation reaction are separated and recovered by distillation, the catalytic system advantageously being recycled after separation by conventional methods, such as separation by settling, electrodialysis, precipitation or filtration.

The cyclohexanol/cyclohexanone mixture can be used in the manufacture of adipic acid by oxidation by nitric acid or can be treated in a dehydrogenation stage to convert the cyclohexanol to cyclohexanone, according to known processes.

Other advantages and details of the invention will become more clearly apparent in the light of the examples given below, which are given solely by way of indication and of illustration.

### EXAMPLE 1

The following:

12.51 g (86.87 mmol) of octanoic acid,

0.527 g (5.38 mmol) of cyclohexanone,

37.88 g (450.9 mmol) of cyclohexane,

0.4431 g (1.245 mmol of Co) of cobalt acetylacetonate,

0.9853 g (5.535 mmol) of N-bromosuccinimide (1.2 mol% with respect to the cyclohexane), are charged to a 125 ml titanium autoclave equipped with means for heating by a heating collar, with a turbine, with gas introduction means and with pressure regulation means.

After closing the reactor, stirring is carried out at 1 000 revolutions per minute, an air pressure (100 bar at 20° C.) is produced and the reactor is heated. The bulk temperature reaches 105° C. in 10 min and this temperature is maintained for a further 3 hours.

After cooling and depressurizing, the reaction mixture comprises a phase comprising cyclohexane and a precipitate.

The mixture is homogenized by addition of acetic acid. The constituents of the mixture are analysed by gas chromatography.

The degree of conversion (DC) of the cyclohexane is: 6.5%.

The selectivity for acids is 32.7%.

The selectivity for the cyclohexanol/cyclohexanone mixture is 51.4%.

The adipic acid/all of the acids molar ratio is 73.2%.

The selectivity for compound X is the yield of this compound calculated with respect to the converted cyclohexane.

### COMPARATIVE EXAMPLE 2

Example 1 is repeated in the same equipment and under the same operating conditions, with introduction of the following reactants:

12.52 g (86.94 mmol) of octanoic acid,

0.5137 g (5.24 mmol) of cyclohexanone,

37.53 g (446.7 mmol) of cyclohexane,

0.4477 g (1.257 mmol of Co) of cobalt acetylacetonate.

The mixture, after homogenization by addition of acetic acid, is analysed by gas chromatography.

The degree of conversion (DC) of the cyclohexane is: 4.0%.

The selectivity for acids is 44.1%.

The selectivity for the cyclohexanol/cyclohexanone mixture is 39.6%.

The adipic acid/all of the acids molar ratio is 76.6%.

### EXAMPLE 3

Example 1 is repeated with a starting reaction mixture exhibiting the following composition:

12.65 g (87.84 mmol) of octanoic acid,

0.5124 g (5.23 mmol) of cyclohexanone,

37.72 g (44 mmol) of cyclohexane,

0.4496 g (1.285 mmol of Co) of cobalt acetylacetonate,

2.0119 g (11.3 mmol) of N-bromosuccinimide (2.5 mol% with respect to the cyclohexane).

The mixture, after homogenization by addition of acetic acid, is analysed by gas chromatography.

The degree of conversion (DC) of the cyclohexane is: 5.4%

The selectivity for acids is 25.9%.

The selectivity for the cyclohexanol/cyclohexanone mixture is 58.9%.

The adipic acid/all of the acids molar ratio is 69.4%.

1. Process for the oxidation of hydrocarbons by an oxidizing agent comprising molecular oxygen, characterized in that it is carried out in the presence of a catalytic system comprising a catalyst based on at least one metal compound and of a cocatalyst comprising at least one imide functional group and corresponding to one of the following general formulae: 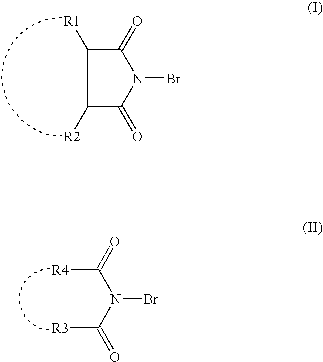
in which:
R1 and R2, which are identical or different, can be hydrogen, an aliphatic, aromatic, cycloaliphatic, arylaliphatic or alkylaromatic hydrocarbonaceous radical which comprises from 1 to 12 carbon atoms and which can comprise heteroatoms, a halogen atom, a hydroxyl group, an alkoxy group, a carboxyl group, an ester group or a carbonyl group, it being possible for the radicals R1 and R2 to be connected to one another to form a cycloaromatic radical, which can comprise several aromatic rings in the condensed or uncondensed form, or a cycloaliphatic radical, which can comprise one or more rings in the condensed or uncondensed form,
R3 and R4, which are identical or different, can be hydrogen or an aliphatic, aromatic, cycloaliphatic, arylaliphatic or alkylaromatic hydrocarbonaceous radical which comprises from 1 to 20 carbon atoms and which can comprise heteroatoms, it being possible for the radicals R3 and R4 to be connected to one another to form a cycloaromatic radical, which can comprise several aromatic rings in the condensed or uncondensed form, or a cycloaliphatic radical, which can comprise one or more rings in the condensed or uncondensed form.

2. Process according to claim 1, characterized in that it is carried out in the gas phase or in the liquid phase.

3. Process according to either of claims 1 and 2, characterized in that a solvent is used in carrying out the process in a liquid medium.

4. Process according to one of the preceding claims, characterized in that the hydrocarbons are saturated aliphatic hydrocarbons or saturated cycloaliphatic hydrocarbons.

5. Process according to claim 4, characterized in that the hydrocarbons are chosen from the group consisting of cyclohexane and cyclododecane.

6. Process according to one of the preceding claims, characterized in that the products obtained are alcohols and/or ketones.

7. Process according to one of claims 1 to 5, characterized in that the products obtained are acids or polyacids.

8. Process according to one of claims 1 to 5, characterized in that the products obtained are a mixture of acids, alcohols and ketones.

9. Process according to claim 3, characterized in that the solvent is a carboxylic acid chosen from the group consisting of acetic acid, glutaric acid, octanoic acid and lipophilic acids.

10. Process according to one of the preceding claims, characterized in that the catalytic system is soluble in the reaction medium.

11. Process according to one of claims 1 to 9, characterized in that the catalytic system is incorporated in a support which is insoluble in the oxidation medium.

12. Process according to one of the preceding claims, characterized in that the catalyst comprises at least one compound of at least one metal element chosen from the group consisting of Cu, Ag, Au, Mg, Ca, Sr, Ba, Zn, Cd, Hg, Al, Sc, In, Tl, Y, Ga, Ti, Zr, Hf, Ge, Sn, Pb, V, Nb, Ta, Cr, Mo, W, Mn, Tc, Re, Fe, Ru, Os, Co, Rh, Ir, Ni, Pd, Pt, lanthanides, such as Ce, and the combinations of these.

13. Process according to one of the preceding claims, characterized in that the metal catalyst comprises a compound of the metal elements chosen from the group consisting of Co and/or Mn and/or Cr and/or Zr, Hf, Ce and/or Zr and/or Hf.

14. Process according to one of the preceding claims, characterized in that R1 and R2 of the formula (I), which are identical or different, represent hydrogen or alkyl radicals chosen from the group consisting of the methyl, ethyl, propyl, isopropyl, butyl, isobutyl, sec-butyl, t-butyl, pentyl, hexyl, heptyl, octyl, decyl and dodecyl radicals and branched radicals, or aromatic radicals chosen from the group consisting of the phenyl, benzyl, naphthyl and toluyl radicals, or cycloalkyl radicals chosen from the group consisting of cyclohexyl, cyclopentyl and cyclooctyl, or alkoxycarbonyl or acyl radicals chosen from the group consisting of methoxycarbonyl, ethoxycarbonyl, propoxycarbonyl, isopropoxycarbonyl, butoxycarbonyl, pentoxycarbonyl and the formyl, acetyl, propionyl, butyryl, valeryl and pivaloyl radicals, or R1 and R2 can be bonded to one another via a single or double bond to form an aromatic or aliphatic ring in the condensed or uncondensed form.

15. Process according to one of claims 1 to 14, characterized in that R3 and R4 of the formula (II), which are identical or different, represent hydrogen or alkyl radicals chosen from the group consisting of the methyl, ethyl, propyl, isopropyl, butyl, isobutyl, sec-butyl, t-butyl, pentyl, hexyl, heptyl, octyl, decyl and dodecyl radicals and branched radicals, or aromatic radicals chosen from the group consisting of the phenyl, benzyl, naphthyl and toluyl radicals, or cycloalkyl radicals chosen from the group consisting of cyclohexyl, cyclopentyl and cyclooctyl, or alkoxycarbonyl or acyl radicals chosen from the group consisting of methoxycarbonyl, ethoxycarbonyl, propoxycarbonyl, isopropoxycarbonyl, butoxycarbonyl, pentoxycarbonyl and the formyl, acetyl, propionyl, butyryl, valeryl and pivaloyl radicals, or R1 and R2 can be bonded to one another via a single or double bond to form an aromatic or aliphatic ring in the condensed or uncondensed form.

16. Process according to one of the preceding claims, characterized in that the cocatalyst is chosen from the group consisting of N-bromosuccinimide, N-bromomaleimide, N-bromohexahydrophthalimide, N,N′-dibromocyclohexane-tetracarboximide, N-bromophthalimide, N-bromotrimellitimide and N,N′-dibromopyromellitimide.

17. Process according to one of the preceding claims, characterized in that the amount of cocatalyst present in the reaction medium is between 0.001 mol and 2 mol of cocatalyst per one mole of hydrocarbon to be oxidized.

18. Process according to claim 17, characterized in that the concentration of catalyst based on metal compounds in the reaction medium is between 0.00001 and 5% (weight% of metal element), preferably between 0.00001% and 2%.
